(19) United States
(12) Patent Application Publication
(10) Pub. No.: US 2025/0001148 A1
(43) Pub. Date: Jan. 2, 2025

(54) MEDICAL DEVICE WITH IMPROVED ACTUATION MECHANISM

(71) Applicant: Novo Nordisk A/S, Bagsvaerd (DK)

(72) Inventors: Noekkvi Steinn Sigurdarson, Koebenhavn N (DK); Finn Hougaard, Koebenhavn K (DK)

(21) Appl. No.: 18/274,106

(22) PCT Filed: Jan. 27, 2022
(86) PCT No.: PCT/EP2022/051967

§ 371 Date: Jul. 25, 2023

Related U.S. Application Data
(60) Provisional application No. 63/142,690, filed on Jan. 28, 2021.

(51) Int. Cl. A61M 31/00

(52) U.S. Cl. CPC A61M 31/007; A61M 31/00

(57) ABSTRACT

A capsule device (100) suitable for ingestion for travelling into a lumen of the gastrointestinal tract of a patient, the lumen having a lumen wall, wherein the capsule device (100) is configured as a self-righting capsule comprising: a) a capsule housing (110, 120) comprising a compartment and an exterior tissue engaging surface (123) defining an exit opening (124) leading from the compartment to the capsule exterior, b) a tissue penetrating member (130) arranged along an axis and configured to be advanced axially from a first position in the compartment through the exit opening (124) and into the lumen wall at a target location, and c) an actuator (140, 150) for coupling to the tissue penetrating member (130), the actuator (140, 150) configured, upon actuation, to move from the a first configuration to a second configuration thereby advancing the tissue penetrating member (130) axially from the first position and into the lumen wall, wherein the actuator (140, 150) comprises a spring (140) formed as a tapered helical tension spring, the spring (140) having a wide first end coupled to the capsule housing (110, 120) and a narrow second end that couples to the tissue penetrating member (130), wherein, in the first configuration, accumulated tension in the spring (140) is maintained, and wherein, upon actuation, accumulated tension in the spring (140) is released thereby moving the actuator (140, 150) towards the second configuration.

The present invention relates to medical devices, including systems for drug delivery, adapted for being inserted into a lumen of a patient and capable of being activated for moving a tissue penetrating member into tissue.

### BACKGROUND OF THE INVENTION

In the disclosure of the present invention reference is mostly made to the treatment of a disease or other condition by delivery of a therapeutic agent, however, this is only an exemplary use of the present invention.

May people suffer from diseases, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering a therapeutic substance into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to oral dosing of active agents and addressing one or more of the above challenges include WO 2018/213600 A1, WO 2020/160399 A1, US 2020/0129441 A1 and WO 2020/157324 A1.

For medical capsules, such as the ones disclosed in the said references, the internal configuration design offers several design challenge trade-offs. For an oral device to be viable, e.g. for delivery of an API in form of a solid needle-shaped API, it needs to deliver an amount of API sufficient for the intended therapy. At the same time, for solid needle-shaped API tablets, the API tablet needs to be delivered reliably into a tissue layer in a depth sufficient to enable systemic uptake. Typically, a large injection force is required to deliver the API tablet at the right depth. Hence, the challenge is to design a device that is small enough to be swallowable, while reliably self-righting and injecting a sufficient amount of API deep enough. Furthermore, low cost and robust performance is essential.

For this kind of actuation mechanism, typical constraints and requirements for obtaining a well-functioning mechanism include the following:
the ability to maintain an actuation member against the load of a drive spring for a prolonged time, even for a drive spring exerting a load of large magnitude.
the actuation member shall be kept in place during dissolving of the dissolvable retaining member in order to maintain the desired acceleration when actuated, and
the dissolvable retaining member preferably needs to be totally dissolved to avoid undissolved pieces that could block the actuation mechanism.

Having regard to the above, it is an object of the present invention to provide a capsule design which is optimized for providing a forceful actuation force, and wherein the outer dimensions of the capsule are minimized.

Another object of the present invention, in embodiments that rely on a self-righting ability for orienting a a tissue penetrating member relative to tissue at a target location of a lumen wall, is to improve the self-righting ability of the self-righting capsule device without compromising actuation force and/or exterior dimensions.

It is furthermore an object of the present invention to provide a medical device for ingestion into a lumen of a patient, and which to a high degree effectively and reliably allows insertion of a tissue penetrating member into a tissue wall of a lumen in a subject.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect, a capsule device is provided which is suitable for ingestion for travelling into a lumen of the gastrointestinal tract of a patient, wherein the lumen has a lumen wall. The capsule device is configured as a self-righting capsule comprising:
a capsule housing comprising a compartment and an exterior tissue engaging surface defining an exit opening leading from the compartment to the capsule exterior,
a tissue penetrating member arranged along an axis and configured to be advanced axially from a first position in the compartment through the exit opening and into the lumen wall at a target location, and
an actuator for coupling to the tissue penetrating member, the actuator having a first configuration and a second configuration, wherein the actuator is configured, upon actuation, to move from the first configuration to the second configuration thereby advancing the tissue penetrating member axially from the first position and into the lumen wall,

wherein the actuator comprises a spring formed as a tapered helical tension spring, the spring having a wide first end coupled to the capsule housing and a narrow second end that couples to the tissue penetrating member, wherein, in the first configuration, accumulated tension in the spring is maintained, and wherein, upon actuation, accumulated tension in the spring is released thereby moving the actuator towards the second configuration.

Previously suggested designs of a self-righting capsule devices, such as disclosed in WO 2020/157324 A1, the proposed capsule devices incorporate a straight cylindrical compression spring configured as the drive spring. In those designs, the actuator comprises a dissolvable pellet trigger arrangement that axially overlaps with the drive spring meaning that the spring needs to fit around the pellet trigger arrangement, i.e., radially outside of the pellet trigger arrangement.

For a given size of the capsule, in an attempt to increase the impact force of the tissue penetrating member, the spring characteristics may be optimized by reducing the coiling diameter and/or increasing the wire diameter. However, the available space left for the trigger system is reduced.

In accordance with the first aspect, by instead providing the drive spring as a tapered helical tension spring, the power of the spring may be increased without having to increase the overall outer dimensions of the capsule device. By utilizing a tension spring in the form of a conical helical spring arranged between the capsule housing and the actuatable component the drive spring now contributes positively to self-orientation. Force/velocity and API payload can now be increased simultaneously without having to increase the overall size of the device.

In some forms the tissue penetrating member has a distal end shaped for penetrating tissue and a proximal end coupled or coupleable to the actuator, wherein when the tissue penetrating member assumes the first position, the distal end is axially separated from the exit opening by a separating distance, thereby enabling the tissue penetrating member to be advanced towards the exit opening by an acceleration stroke corresponding to the separating distance. By increasing the acceleration stroke, more energy for penetrating mucosal tissue will be available.

In different embodiments, the acceleration stroke is between 1 mm and 8 mm, such as between 2 and 7 mm, and such as between 3 mm and 5 mm.

In further embodiments, a guide structure is arranged between the capsule housing and the tissue penetrating member, the guide structure comprising a central portion being configured for cooperating with the distal end of the tissue penetrating member thereby guiding the distal end for movement along the axis as the distal end of the tissue penetrating member moves towards the exit opening.

In some forms the guide structure is configured for guiding the distal end of the tissue penetrating member along a major portion of the acceleration stroke, such as more than 60 percent, such as more than 70 percent, such as more than 80 percent, and such as more than 90 percent of the acceleration stroke.

In still further forms, the central portion is formed to engage with the tissue penetrating member and to allow the tissue penetrating member to escape through the central portion as the tissue penetrating member advances through the exit opening of the capsule housing.

The guide structure may be formed so that it comprises a peripheral portion coupled to the capsule housing, wherein the central portion is configured for axial movement, and wherein the guide structure comprises at least one flexible connector connecting the peripheral portion with the central portion.

In the first configuration, the at least one flexible connector may be formed to assume a shape having a portion that is inclined relative to the axis so as to extend from the peripheral portion to the central portion in a radially inwards and proximal direction.

In exemplary embodiments the flexible connector is so configured that, when the distal end of the tissue penetrating member assumes an axial position at the exit opening, said portion of the flexible connector has been shifted to assume a shape being inclined relative to the axis so as to extend from the peripheral portion to the central portion in a radially inwards and distal direction.

In some forms, the guide structure is formed at least in part from an elastomeric material, such as rubber, and wherein, in the first configuration, the at least one flexible connector is conically shaped.

The spring may be provided so that, in the first configuration, the spring assumes a conical shape extending along the axis.

In some forms the actuator comprises an actuation member. The actuation member may in some embodiments be configured to couple relative to the tissue penetrating member, such as to hold the tissue penetrating member during movement of the actuator between the first configuration and the second configuration.

In some exemplary embodiments, the spring is configured for moving the actuation member initially in the distal direction and subsequentially in the proximal direction.

The spring comprises a plurality of windings. For some embodiments, when in the first configuration, at least one of the plurality of windings encircle the tissue penetrating member in an axially overlapping manner.

In other embodiments, when in the first configuration, a majority of the plurality of windings encircle the tissue penetrating member in an axially overlapping manner.

In some forms, the tissue penetrating member is a solid delivery member formed partly or entirely from a preparation comprising a therapeutic payload, and wherein the preparation is made from a dissolvable material that dissolves when inserted into tissue of the lumen wall to at least partly release the therapeutic payload into the blood stream.

In some embodiments, when in the first configuration, the second narrow end of the spring is located proximally to the first wide end of the spring, whereas, in the second configuration, the second narrow end of the spring is located distally to the first wide end of the spring.

The actuator may be provided as an actuation mechanism that comprises a firing mechanism configured for actuating the actuation mechanism to enable the drive spring to drive the actuation member upon occurrence of a pre-defined condition. In some embodiments, the predefined condition is provided as one or more of a pre-defined condition in the lumen, a set of pre-defined conditions in the lumen, or a signal received by the capsule device and emitted by an external controlling device. In still further embodiments, the firing mechanism comprises an environmentally sensitive mechanism. The firing mechanism may be configured so that in the pre-actuation configuration the firing mechanism retains the actuator in the first configuration, and in a firing configuration releases the actuator for enabling the spring to drive the actuation member from the first configuration to the second configuration.

In some embodiments the firing mechanism is provided as a dissolvable pellet trigger arrangement wherein a dissolvable pellet is configured to releasably retain the actuation member relative to the capsule housing and against the mechanical load provided by the spring. The dissolvable pellet may be configured for being exposed to a biological fluid, such as a gastric fluid of the GI-tract, to enable dissolution of the dissolvable pellet thereby actuating the actuation mechanism, e.g. by releasing the actuation member relative to the capsule housing.

In some embodiments, non-limiting examples of firing mechanisms are provided as dissolvable pellet trigger arrangements and may utilize any of the principles disclosed in WO 2020/157324 A1.

In still further embodiments according to the first aspect, the capsule device is defined by:
a capsule housing having proximal and distal ends, the capsule housing comprising a plurality of retaining portions, and
an actuation mechanism operable between a pre-actuation configuration and an actuating configuration,
wherein the actuation mechanism comprises:
an actuation member arranged within the capsule housing and configured for distal movement along an axis from a first position towards a second position, wherein the actuation member comprises a base portion and a plurality of latch arms extending proximally therefrom, wherein each latch arm defines radially outwards and radially inwards facing latch surfaces, and wherein each latch arm is radially deflectable,
a drive spring having helical windings arranged along the axis, the drive spring having a first end coupled to the housing and an opposite second end coupled to the actuation member, wherein the drive spring in the pre-actuation configuration is strained to exert a load onto the actuation member for driving the actuation member towards the second position, and
a dissolvable latch support,
wherein the dissolvable latch support is arranged centrally on-axis, wherein the retaining portions are arranged coaxially around the dissolvable latch support, and wherein the latch arms are arranged radially in between,
wherein, in the pre-actuation configuration, the radially outwards facing latch surfaces engage with respective retainer portions in a latching engagement, and the radially inwards facing latch surfaces are in supporting engagement with respective radially outwards facing surfaces of the dissolvable latch support thereby restricting the latch arms from moving radially inwards and preventing release of the latching engagement, and
wherein, in the actuating configuration where the dissolvable latch support has become at least partially dissolved, the latch arms are allowed to move radially inwards thereby releasing the latching engagement and allowing the drive spring to move the actuation member towards the second position.

In some such capsule embodiments, in the pre-actuation configuration, the latch arms extend proximally and radially outwards from the base portion of the actuation member so that the radially inwards facing latch surface of each latch arm is inclined relative to the axis, and wherein the respective radially outwards facing surfaces of the dissolvable latch support are inclined thereby supporting the respective latch arm along the radially inwards facing latch surface.

By configuring the latch mechanism with latch arms that extend proximally and radially outwards from the base portion so that the radially inwards facing latch surface of each latch arm is inclined relative to the axis this enables, in the actuating configuration, the latch arms to collapse radially toward each other in a manner where the latch arms only take up little space in the radial direction. This enables the retaining portions associated with the capsule housing to be formed with a radially minor dimension.

Compared to latch mechanisms of the prior art, the proposed latch mechanism offers improvements to the load bearing surfaces, and the risk of creep in the latch mechanism components is reduced.

Furthermore, the solution enables more effective wetting of the dissolvable latch support resulting in improved precision for timely releasing of the latching engagement.

In some embodiments, when in the pre-actuation configuration, the dissolvable latch support is primarily acted upon by compression forces exerted onto the dissolvable latch support by the latch arms being urged radially towards the dissolvable latch support.

In some embodiments, in the pre-actuation configuration, at least one pair of a latch arm and a retainer portion is structured to maintain, i.e. releasably retain, the actuation member.

In some forms of the capsule device, when assuming the pre-actuation configuration, the spring exerts a load on the actuation member, and the at least one pair of a latch arm and a retainer portion retains the actuation member relative to the base member against the load exerted by the spring.

In other forms, when the capsule device is taken into use, the spring will not initially exert a load on the actuation member, but may be operated, such as by a user-initiated step, to provide said load.

In some embodiments the plurality of latch arms are provided as two radially opposed latch arms arranged in a v-shaped configuration, and wherein the dissolvable latch support member is generally wedge shaped or shaped as a cone. In other embodiments, the number of latch arms may be three, four, five or more, wherein the latch arms are distributed around the axis.

The capsule device may be formed so that a fluid ingress opening is provided in the capsule housing proximal end, wherein the dissolvable latch support is disposed in the fluid ingress opening to enable wetting of the dissolvable latch support.

In some embodiments, the plurality of retaining portions each comprise an inclined surface with a surface normal pointing proximally and radially inwards, and wherein the radially outwards facing latch surface of each latch arm comprises a correspondingly inclined surface.

The capsule housing defines an exterior surface. The inclined surfaces of the retaining portions may be formed to intersect with the capsule housing exterior surface.

The engagement interfaces between the radially outwards facing latch surfaces and the respective retainer portions may in certain embodiments be provided either as planar interfaces or single curvature interfaces, such as conically shaped interfaces.

In further embodiments, the engagement interfaces between the radially inwards facing latch surfaces and the respective radially outwards facing surfaces of the dissolvable latch support may be provided as planar interfaces or single curvature interfaces, such as conically shaped interfaces.

The exterior of the capsule housing defines a housing extreme proximal end surface. In some embodiments, the dissolvable latch support defines a dissolvable latch support proximal end surface, and wherein, in the pre-actuation configuration, the dissolvable latch support proximal end surface is located proximally relative to the housing exterior extreme proximal end. In alternative embodiments the dissolvable latch support proximal end surface is located distally within 2 mm, such as within 1.5 mm such as within 1.0 mm, such as within 0.5 mm from the housing exterior extreme proximal end.

In some forms, the tissue penetrating member comprises a therapeutic payload.

In some further forms the tissue penetrating member is provided as a solid delivery member formed partly or entirely from a preparation comprising a therapeutic payload, and wherein the preparation is made from a dissolvable material that dissolves when inserted into tissue of the lumen wall to at least partly release the therapeutic payload into the blood stream.

The therapeutic payload may in other embodiments be in the form of an encapsulated solid, a liquid, a gel or a powder, or any combination thereof and configured for delivery through a delivery member which defines the tissue penetrating member.

In some embodiments, the capsule device comprises a delivery member being associated with the therapeutic payload, the delivery member being configured for insertion into the lumen wall to deliver at least a portion of the therapeutic payload. The delivery member may have an outer shape as a needle. However, in alternative embodiments, different shapes for the delivery member may be provided.

In still other embodiments the delivery member is an injection needle having a longitudinal lumen extending within the injection needle, wherein the therapeutic payload is provided as a liquid, gel or powder being expellable through the injection needle from a reservoir accommodated within the capsule device.

In some forms the capsule device defines an ingestible device suitable for swallowing by a patient and travelling into a lumen of a gastrointestinal tract of a patient, such as the stomach or the small intestines. The capsule hosing of the device may be shaped and sized to allow it to be swallowed by a subject, such as a human.

By the above arrangements an orally administered drug can be delivered safely and reliably into the stomach or intestinal wall of a living mammal subject, such as a human.

In some embodiments, the actuation member may define a hub configured for moving the tissue penetrating member from a first position stored within the capsule housing to a second position different than the first position so that, in the second position, at least a part of the tissue penetrating member is brought into direct engagement with tissue exterior to the capsule housing.

In some forms the actuator comprises an actuation member that moves from a first position to a second position as the actuator moves from the first configuration to the second configuration.

In some embodiments, the actuation mechanism is operable from a pre-actuation configuration, through an actuating configuration and end in an actuated configuration. The actuating configuration may be referred to as an intermediary configuration wherein the actuation mechanism releases the actuation member for movement towards the actuated configuration.

In some embodiments, in the pre-actuation configuration, the tissue penetrating member is attached to the hub.

In other embodiments, in the pre-actuation configuration, the tissue penetrating member is not engaged by the hub, but wherein during the hub moving from the first position to the second position, the hub initially engages the tissue penetrating member, and subsequently moves the tissue penetrating member so that at least a part of the tissue penetrating member is inserted into tissue exterior to the capsule housing.

The hub may be configured to carry or push one or more of a therapeutic payload, a delivery member, and a sensor from a first position relative to the capsule housing to a second position relative to the capsule housing.

In particular forms the hub itself forms one or more of a therapeutic payload, a delivery member, and a sensor that moves from a first position relative to the capsule housing to a second position relative to the capsule housing.

In some forms the hub comprises an interface portion, wherein the tissue penetrating member attaches relative to the interface portion of the hub.

In such embodiments the hub may be actuated to move the hub from a first position to a second position, and wherein the tissue penetrating member is configured for detachment relative to the interface portion of the hub when the hub assumes the second position.

In further exemplary embodiments the capsule device defines a capsule device comprising a capsule housing having an outside shape formed as a rounded object and defining an exterior surface. The capsule device further comprises a solid delivery member having a needle or dart-shaped form and being formed partly or entirely from a preparation comprising a therapeutic payload, wherein the preparation is made from a dissolvable material that dissolves when inserted into tissue of the lumen wall. The actuation member is formed as a hub that comprises an interface portion, wherein the solid delivery member is held or attached relative to the interface portion of the hub. The capsule device is configured as a self-righting capsule having a geometric center and a center of mass offset from the geometric center along the axis, wherein when the capsule device is supported by the tissue of the lumen wall while being oriented so that the centre of mass is offset laterally from the geometric center the capsule device experiences an externally applied torque due to gravity acting to orient the capsule device with the axis oriented along the direction of gravity to enable the solid delivery member to interact with the lumen wall at the target location. Upon entering into the actuating configuration, the hub moves along the axis thereby inserting the solid delivery member into tissue. Subsequent to insertion, the solid delivery member may at least partially dissolve and release one or more therapeutic agent(s) into the tissue.

Non-limiting examples of a self-righting capsule device may include devices configured for actuation when the device is located in the stomach lumen of a patient. Other non-limiting examples may include devices configured for actuation when the device is located in the small intestines or the large intestines.

Whereas this disclosure mainly refers to drug delivery from a capsule device to a lumen wall, the invention in its broadest aspect is not limited to drug or substance delivery. The capsule device according to the present invention may be configured for other uses. Non-limiting uses include obtaining one or more samples from a body lumen, e.g. by including a sample taking device for introducing a sample from a body lumen wall into the capsule device, and delivering a monitoring or analysis device, e.g. by disposing or positioning a sensor device from the capsule device into the lumen wall.

As used herein, the terms “drug” “therapeutic agent”, “payload” or “therapeutic payload” is meant to encompass any drug formulation capable of being delivered into or onto the specified target site. The drug may be a single drug compound or a premixed or co-formulated multiple drug compound. Representative non-limiting drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein:

FIGS. 1a and 1b each shows a cross-sectional front view of a first embodiment capsule device 100 in accordance with the invention, the device assuming a pre-actuation configuration and an actuated configuration, respectively,

FIG. 2 is a perspective partly cut view of the first embodiment capsule device 100 in the pre-actuation configuration,

FIGS. 3a and 3b are two different perspective views of a hub 150 of capsule device 100 according to the first embodiment,

FIGS. 4a and 4b are two different perspective views of a dissolvable latch support 160 of capsule device 100 according to the first embodiment,

FIGS. 5a through 5c each shows a cross-sectional front view of a second embodiment capsule device 100′ in accordance with the invention, the device assuming a pre-actuation configuration, an intermediate configuration and an actuated configuration, respectively,

FIG. 6 is a perspective partly cut view of the second embodiment capsule device 100′ in the pre-actuation configuration,

FIGS. 7a and 7b are two different perspective views of a hub 150 of capsule device 100′ according to the second embodiment, and

FIGS. 8a and 8b are two different perspective views of a dissolvable latch support 160 of capsule device 100′ according to the second embodiment.

In the figures representing views of the different embodiments like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g., manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

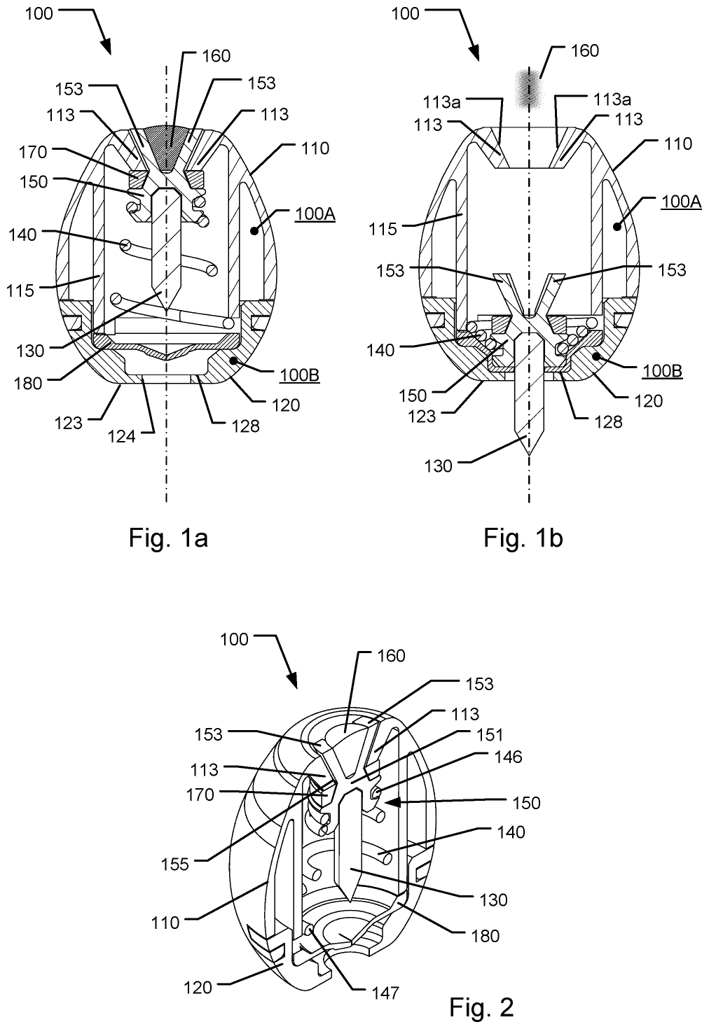

With reference to FIGS. 1a, 1b and 2 a first embodiment of a drug delivery device in accordance with an aspect of the invention will be described, the embodiment being designed to provide a capsule device 100 having a desired actuation configuration for deployment of a solid dose from a solid dose capsule device. The disclosed embodiment relates to a capsule device 100 suitable for being ingested by a patient to allow the capsule device to enter the stomach lumen, subsequently to orient relative to the stomach wall, and finally to deploy a solid dose payload for insertion at a target location in tissue of the stomach wall. Generally, at least a portion of the solid dose payload penetrates the tissue of the subject and at least a portion of a therapeutic agent within the payload dissolves into the tissue for uptake in the blood stream of the subject. For the capsule device 100 the general principle for orienting the capsule relative to the stomach wall may utilize any of the principles disclosed in WO 2018/213600 A1. The state shown in FIGS. 1a and 2 represents a pre-actuation configuration prior to swallowing of the capsule, whereas the state shown in FIG. 1b represents an actuated configuration, i.e., corresponding to a post-delivery state.

The ingestible self-righting capsule device 100 comprises a first portion 100A having an average density, a second portion 100B having an average density different from the average density of the first portion 100A. The capsule device 100 accommodates a tissue penetrating member forming a payload portion 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article is a gomboc shape, i.e., a gomboc-type shape that, when placed on a surface in any orientation other than a single stable orientation of the shape, then the shape will tend to reorient to its single stable orientation.

The capsule device shown includes an upper (proximal) capsule housing 110 which mates and attaches to a lower (distal) capsule housing 120. The upper capsule housing 110 and the lower capsule housing 120 together forms the capsule housing of the device. In the shown embodiment upper capsule housing 110 and lower capsule housing 120 are mounted relative to each other by way of a snap engagement. The capsule housing parts 110/120 define a shell having an interior hollow which accommodates the payload portion 130 and an actuation and propulsion mechanism. The latter comprises an energy source in the form of a pre-strained drive spring 140, and an actuation member in the form of hub 150 which holds and drives forward the payload portion 130 for payload delivery upon release of energy from the drive spring 140. The payload portion 130 is oriented along an actuation axis and configured for movement along the actuation axis. In the shown embodiment, the upper and lower capsule housing parts 110, 120 form generally rotation symmetric parts with the axis of symmetry arranged along the actuation axis. In the drawings, the device is oriented with the actuation axis pointing vertically, and with the payload portion 130 pointing vertically downwards towards an exit opening/exit hole 124 arranged centrally in the lower capsule housing 120, the exit hole allowing the payload portion 130 to be transported through exit hole and moved outside the capsule device 100. The lower capsule housing part 120 includes a tissue engaging surface 123 which is formed as a substantially flat lower outer surface surrounding the exit hole 124.

In the shown embodiment in FIG. 1a, the distal pointed end of the payload portion 130 is situated axially a distance relative to a point on the actuation axis where the axis intersects with the tissue engaging surface 123, this distance being referred to as the separating distance. This enables the tissue penetrating member to be advanced towards the exit opening 124 by an acceleration stroke corresponding to the separating distance.

Regarding suitable materials for the capsule housing for the embodiment shown in FIGS. 1a and 1b, the upper capsule housing 110 may suitably be made from a low-density material, such as polycaprolactone (PCL), whereas the lower capsule housing 120 may be suitably made from a high-density material, such as 316L stainless steel. In other embodiments, other materials may be used.

In the shown embodiment, due to the density distribution of the entire capsule device 100, and due to the outside shape of the device, when the capsule device is supported on a wall such as a tissue wall, and being subjected to gravitational forces, the capsule device 100 will tend to orient itself with the actuation axis substantially perpendicular to the surface (e.g., a surface substantially orthogonal to the force of gravity, a surface of a tissue such as the wall of the gastrointestinal tract). Hence, the capsule device seeks to orient relative to the direction of gravity so that the tissue engaging surface 123 faces vertically downward.

The interior of the upper capsule housing 110 includes a mounting structure provided as an inner sleeve 115 which extends concentrically with the actuation axis from the upper part of the upper capsule housing 110 towards a proximally facing bottom surface formed in the lower capsule housing 120.

Further, in the shown embodiment, a hub retaining structure 113 is provided as an inwardly extending round-going flange that is arranged concentrically with the actuation axis and which extends radially inwards relative to the inner sleeve 115 from the upper capsule housing 110 and downwards along the actuation axis. The hub retaining structure 113 serves as a retaining geometry for releasably retaining the hub 150 against the drive force emanating from a strained drive spring 140 arranged within the capsule. Referring mainly to FIG. 1b, in the shown embodiment, the hub retainer structure 113 provides a conical retainer surface 113a wherein a surface normal to the conical retainer surface 113a points proximally and radially inwards. In the shown embodiment the conical retainer surface 113a interfaces directly with the exterior rounded surface at a proximal end portion of upper capsule housing 110. In the shown first embodiment of capsule device 100, the conical retainer surface 113a of hub retaining structure 113 forms an angle of 25 deg. relative to the actuation axis.

At the distal most portion of the conical retainer surface 113a, a central opening is formed at the centre thereof. The central opening is dimensioned so that the hub 150 is movable axially through the central opening when the hub assumes a released state but wherein the hub 150 cannot move axially through the central opening when the hub 150 assumes a state corresponding to the actuation configuration. The conical retainer surface 113, at the locations where upper edges of the conical retainer surface 113 intersect with the exterior surface of the capsule housing 110, the structure defines a fluid ingress opening allowing gastric fluid to enter into contact with a fluid operated actuation mechanism.

In the first embodiment shown in FIGS. 1a and 1b, payload portion 130 defines a solid delivery member formed entirely or partly from a preparation comprising the therapeutic payload. In the shown embodiment, the solid delivery member is formed as a thin cylindrical rod which is shaped to penetrate tissue of the lumen wall, the cylindrical rod having a tissue penetrating end, i.e. a tip portion, and a trailing end opposite the tissue penetrating end. In the shown embodiment, the tissue penetrating end of the rod is pointed to facilitate easy insertion into mucosal tissue of the lumen wall whereas the trailing end, in the shown embodiment, defines a truncated cylinder cut off by a 90-degree cut. A non-limiting example of a drug suitable for delivery by capsule device 100 is dried compressed API, such as insulin.

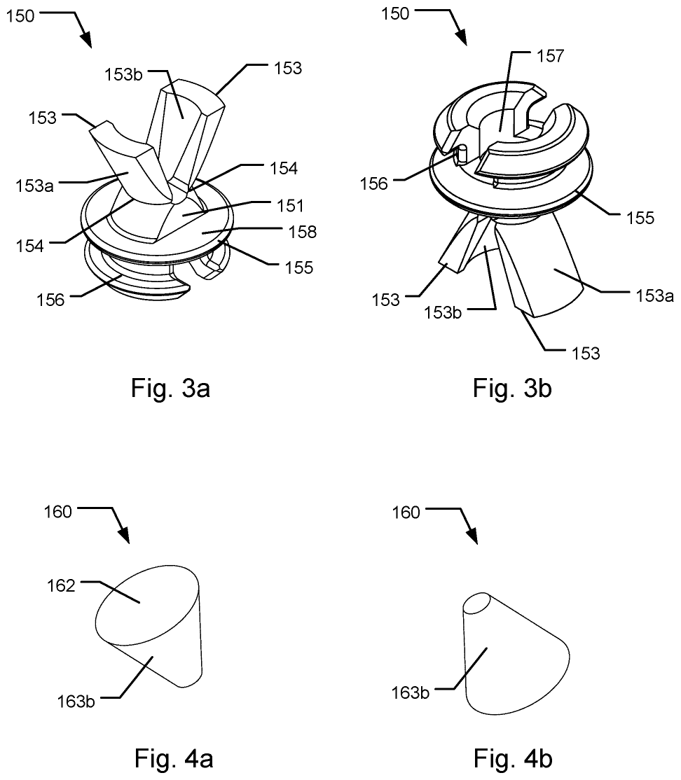

Referring mainly to FIGS. 3a and 3b, the hub 150 comprises an upper retaining part 151 configured for releasably retaining the hub relative to the capsule housing and a lower interface part 155 configured for holding the trailing end of the payload portion 130 in place. In the shown embodiment, the lower interface part 155 includes a downward open bore 157 that receives the trailing end of the payload portion 130 in a way so that the payload portion 130 is firmly attached within the bore. The lower interface part 155 further defines an annular spring seat, in the following referred to as the ‘second spring seat’ 146.

The upper retaining part 151 of the hub 150 forms a base portion which at a proximal end connects with two latches provided in the form of two independently deflectable latch arms 153. In the state shown in FIG. 1a, the latch arms 153 are symmetrically arranged around the actuation axis. Each latch arm extends from the retaining part 151 at an angle with respect to the axis so that they extend inclined proximally and radially outwards from the base portion. The two latch arms are thus configured in a v-shaped configuration. Each latch arm 153 is resiliently movable in the radial inwards direction by a swivelling movement relative to the upper retaining part 151. The latch arms 153 each defines a radially outwards facing latch surface 153a configured to engage with respective portions of the conical retainer surface 113a in a latching engagement. Each of the latch arms 153 further includes a radially inwards facing latch surface 153b configured for cooperating with a centrally disposed dissolvable latch support 160, see FIGS. 1a and 4a/4b. In the embodiment shown all the surfaces 153a and 153b are formed as conical surfaces.

In the shown first embodiment, in the pre-actuation configuration shown in FIG. 1a, the angle of inclination of the radially outwards facing latch surface 153a forms an angle of 25 deg. relative to the axis whereas the angle of inclination of the radially inwards facing latch surface 153b forms an angle of 22 deg. relative to the axis. Hence, from the base portion 151 towards the free end of the latch arms, the lateral thickness of each latch arm increases gradually towards a larger lateral thickness at the free end. In other embodiments, different angles of inclination may be used for surfaces 153a and 153b. Also, the angle of inclination may be the same for surfaces 153a and 153b or the difference in angles of inclination between surfaces 153a and 153b may be even larger.

In the shown embodiment of hub 150, the latch arms 153 connect to the base portion of upper retaining portion 151 by means of a hinge section allowing the two latch arms, relative to the positions they assume in FIG. 1a, to become deflected radially inwards towards a collapsed position wherein the latch arms either touch each other or are positioned with a minor radial spacing between them. Hence, upon deflecting radially inwards, the deflectable latch arms are allowed to release from conical retainer surface 113a thereby allowing the hub 150 to move distally relative to the capsule housing 110/120 through the central opening formed in the conical retainer surface 113a.

It is to be noted that FIGS. 3a and 3b show the hub 150 in the state corresponding to the pre-actuation configuration shown in FIG. 1a. However, prior to assembly of the capsule device 100, i.e. prior to insertion of hub 150 into upper capsule housing 110, the latch arms may assume a different orientation when they assume a relaxed state. Hence, during manufacturing of the hub 150, which may be manufactured by injection molding, such as by molding using a high-strength thermoplastic material such as polyoxymethylene (POM) or similar material, the latch arms 153 may be formed so that, when the latch arms are in a relaxed state the latch arms assume the collapsed position wherein the arms are collapsed radially towards each other. This enables easy assembling of the capsule device, by allowing the hub 150 to be inserted in the proximal direction through the central opening formed in the conical retainer surface 113a without requiring a separate step of moving the latch arms towards the collapsed state.

FIGS. 4 and 4b show two perspective views of the dissolvable latch support 160. In the shown embodiment, the dissolvable latch support 160 is formed as a generally cone-shaped member sized to be inserted between the two latch arms 153 in a wedging manner forcing the latch arms 153 in intimate contact with the conical retainer surface 113a of the hub retainer structure 113. In the shown embodiment, the conical surface 163b matches the surface 153b of the latch arms with an angle of inclination of 22 degrees relative to the central axis of the cone.

For the dissolvable latch support 160, different forms and compositions may be used. Nonlimiting examples include pellets made from Sorbitol or Microcrystalline cellulose (MCC). Other non-limiting examples include injection moulded Isomalt pellets, compressed granulate Isomalt pellets, compressed pellets made from a granulate composition of Citrate/NaHCO₃, or compressed pellets made from a granulate composition of Isomalt/Citrate/NaHCO₃.

Such dissolvable latch support will become disintegrated when subjected to a liquid such as gastric juice of the GI-tract. By carefully selecting the composition, the geometry of the dissolvable latch support and optionally exposure channels to ensure wetting of the dissolvable latch support, the release time can be controlled to occur within a chosen time delay after swallowing of the capsule device 100.

The first embodiment capsule device 100 additionally comprises a pair of sealing elements 170, 180 for maintaining the tissue penetrating member, i.e. the payload portion 130, fluidically isolated from the environment external to capsule device 100 prior to actuation. In the shown embodiment, an upper sealing element 170 formed as a ring of soft pliable material, such an elastomeric material, is inserted between the lowermost annular surface of the conical retainer surface 113a and an annular proximal facing flange surface 158 of the hub 150.

The further sealing element, i.e. the lower sealing element 180, forms a fluidic gate configured to maintain the exit hole 124 fluidically blocked prior to actuation. In the shown embodiment, the sealing element 180 comprises an elastomeric seal member having a generally disc shaped form. An outer periphery of the sealing element 180 is mounted below the lowermost winding of the drive spring 140 and clamped above an annular proximally facing surface of lower capsule housing 120. As disclosed in US 2020/0129441 A1 the central area of the sealing element 180 may comprise a fluidic gate formed to provide a self-sealing valve, such as formed by one or more thin cuts (e.g., one or more thin slits) that extend partially or completely through a thickness of the fluidic gate.

The sealing elements 170 and 180 thus cooperate to form a compartment internally in capsule device 100 that serves, prior to actuation, to maintain the payload portion 130 fluidically isolated from biological fluid externally to capsule device 100 but allows the payload portion to penetrate easily through sealing element 180 at the time of actuation for payload delivery into tissue.

During assembly, after the latch arms of the hub 150 has been inserted fully proximally through the central opening formed in the conical retainer surface 113a and with the sealing element 170 clamped between the hub 150 and the upper capsule housing 110, the latch support 160 is forced axially in the distal direction in between the latch arms 153. Due to the conical interface between the dissolvable latch support 160 and the latch arms 153, the dissolvable latch support 160 is allowed to be moved distally in a wedging action while the latch arms 153 become spread radially outwards into engagement with the conical retainer surface 113a. At the end of this assembly step, the wedging action provides stiction between the dissolvable latch support 160 and the latch arms 153 resulting in mounting engagement where the dissolvable latch support 160 remains fixedly attached to the latch arms 153. This enables safe storage and handling without the risk of dissolvable latch support 160 becoming accidentally dismounted from the latch arms 153. As seen in FIG. 1a, the top surface 162 of dissolvable latch support is located substantially flush with the proximal opening defined by conical retainer surface 113a. This serves to ensure adequate wetting of the dissolvable latch support 160 when submerged into gastric juice. In other embodiments, the top surface 162 may either be located proximally or located distally to the extreme proximal end of the upper capsule housing 110.

With regard to the above-mentioned drive spring 140, in capsule device 100, the drive spring is provided in the form of a pre-strained helical tension spring arranged coaxially with the actuation axis inside the capsule housing. The drive spring, in the pre-actuation configuration shown in FIG. 1a, defines a wide first end 147 mounted relative to the capsule housing 110/120 and a narrow second end 146 mounted onto the actuator 150, i.e. such that the drive spring 140 defines a large diameter portion at the first end 147 and a narrow diameter portion at the second end 146, the large diameter portion being larger than the narrow diameter portion. In the pre-actuation configuration, the first end 147 is located distally to the second end 146. In the embodiment shown, the drive spring 140 is provided as a conical tapering spring which exclusively is arranged for operating in tension mode. In this embodiment the first end 147 of the drive spring is mounted at the extreme distal portion of the capsule, i.e. at an axial location close to the exit hole 124, and thus accommodated in a space surrounded by the lower capsule housing 120.

The first end 147 of drive spring 140 is seated against a first spring seat arranged in the distal end portion of the capsule device 100. In the shown embodiment, the first spring seat is formed by a distal end face of inner sleeve 115. The distal end face of inner sleeve 115 is arranged with a slight axial spacing relative to a proximally facing surface of the lower capsule housing 120. A substantial portion of the lower-most winding of the drive spring 140 defines a diameter comparable with the diameter of inner sleeve 115. Inner sleeve 115 is formed with a distal end face disposed with some distance relative to a proximal facing end surface of lower capsule housing 120 allowing said portion of the lower-most winding of the drive spring 140 to radially overlap with the inner sleeve 115 and in this way become clamped in a circumferential slot between inner sleeve 115 and lower capsule housing 120.

The second end 146 of drive spring 140 is seated against a second spring seat 156 formed by the lower interface part 155 of the hub 150. As part of assembling the capsule device 100 the drive spring 140 has been energized by axially tensioning the drive spring 140 between the two spring seats. Hence, the hub 150 is initially under tension load from drive spring.

In the embodiment shown, prior to final assembly and with the drive spring 140 arranged along the actuation axis but with the drive spring assuming a non-energized state, the second narrow end 146 of the spring would be positioned distally to the first wide end 147 of the spring. However, during assembly wherein the drive spring 140 is increasingly strained, the second narrow end 146 will be moved proximally relative to the first wide end 147 to enter into the pre-actuation state shown in FIG. 1a wherein the drive spring is fully energized.

Turning now to the operation of the capsule device 100, reference is initially made to FIGS. 1 and 2 which show the initial state which represents the state the capsule device assumes during storage or just after ingestion. In this state, the actuator 150 assumes the pre-actuation configuration where the two latch arms 153 are maintained in the shown position by engagement with radially outwards facing surfaces 163b of the dissolvable latch support 160 engaging the radially inwards facing latch surface 153b of the latch arms. As a result, the radially outwards facing latch surface 153a of the latch arms are kept in engagement with the conical retainer surface 113a preventing the latch arms from sliding relative to the conical retainer surface. As the latch arms 153 are trapped proximally to the conical retainer surface 113a the hub 150 cannot be moved distally even though the drive spring 140 exerts its full tensile load onto the actuator 150.

The upper sealing element 170 engages the flange 158 as well as the lower surface of hub retainer structure 113 to keep this interface fluid tight. Also, the lower sealing element 180 keeps the exit hole 124 fluid tight.

After ingestion of capsule device 100, the capsule device quickly sinks to the bottom of the stomach. Upon being supported by the stomach wall, due to the self-righting ability of the capsule device, the capsule device will quickly reorient to have its tissue interfacing surface 123 engaging the tissue stomach wall with the firing axis of the capsule device oriented virtually vertical, i.e. with the payload portion 130 pointing downwards. Dissolvement of dissolvable latch support 160 has begun due to exposure to gastric fluid. This is represented in FIG. 1b in connection to reference 160. The support from dissolvable latch support 160 against the latch arms 153 will cease at a specific time after swallowing. The load of the drive spring 140 will cause the latch arms 153 to be gradually deflected radially inwards thereby allowing the latch arms to slide off from engagement with the conical retainer surface 113a. At some point in time the latch arms 153 will reach their collapsed position where after the hub 150 with the payload portion 130 will become released from the hub retainer structure 113. This state corresponds to the actuating configuration. As drive spring 140 exerts tension onto hub 150, the hub and the payload portion 130 are caused to travel unhindered towards the exit hole 124 with the payload portion penetrating the lower sealing element 180 and further into mucosal tissue at the target location. In the actuated configuration, as shown in FIG. 1b, a proximally facing hub stop surface 128 arranged at the bottom part of capsule housing 120 prevents the hub 150 from moving further distally. It is seen that the second end of the spring 146, in the course of the delivery stroke, has been travelling axially slightly distal to the first end of the spring 147. Due to the tapering nature of the tension spring, the hub will have a marked tendency of self-centring to travel along the actuation axis without requiring further radial guidance relative to the capsule housing.

In situation of intended use, the payload portion 130 is inserted into tissue of the lumen wall where it will anchor generally in a direction along the actuation axis. As discussed above, depending on the specific design of the capsule device, the payload portion 130 may be released actively from the remaining parts of the capsule at the end of the insertion stroke. When the capsule device 100 has delivered the intended dose the capsule will release relative to the deposited payload portion 130 which remains inside the tissue wall for release of therapeutic agent into the blood stream of the subject.

Although not shown in the embodiments disclosed herein, any of the embodiments may be modified to include a mechanism for separating the payload portion 130 from the hub 150 upon the assembly of the payload portion 130 and the hub 150 arriving at the most distal position in the capsule housing. Suitable non-limited principles may include the principles disclosed in WO 2020/245448 A1 wherein a ram (similar to a hub) becomes tilted at the end of the insertion stroke for detaching the tissue inserted portion of the delivery member from the ram.

Alternatively, the capsule may be held stationary for a prolonged time allowing the payload portion 130 to release a therapeutic agent into the blood stream of the subject as the capsule is held stationary relative to the tissue. In any of these cases, subsequently to drug delivery, the remaining parts of the capsule device will travel out through the digestive system of the user and be disposed of.

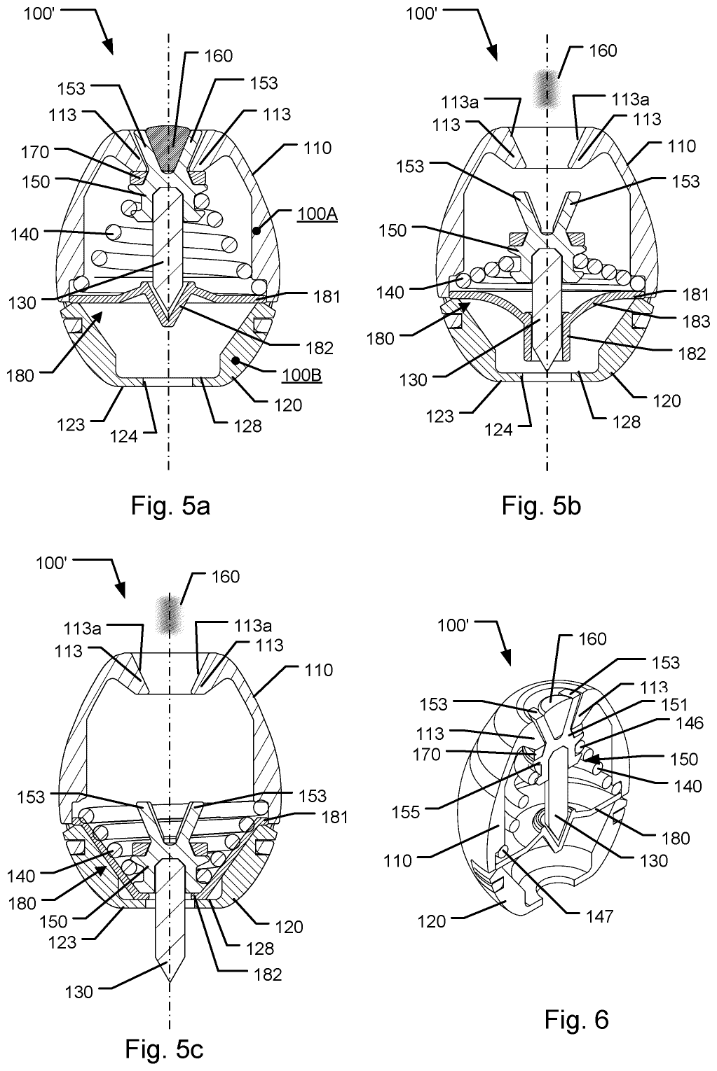

Switching now to a second embodiment of a capsule device 100′ in accordance with the invention, reference is made to FIGS. 5a-5c, 6, 7a-7b and 8a-8b. The capsule device 100′ shares many constructional features with the corresponding features of capsule device 100 described above, and the general principle of operation is the same. However, the drive spring 140 has been modified and a guide system for guiding the payload portion during the delivery stroke has been introduced.

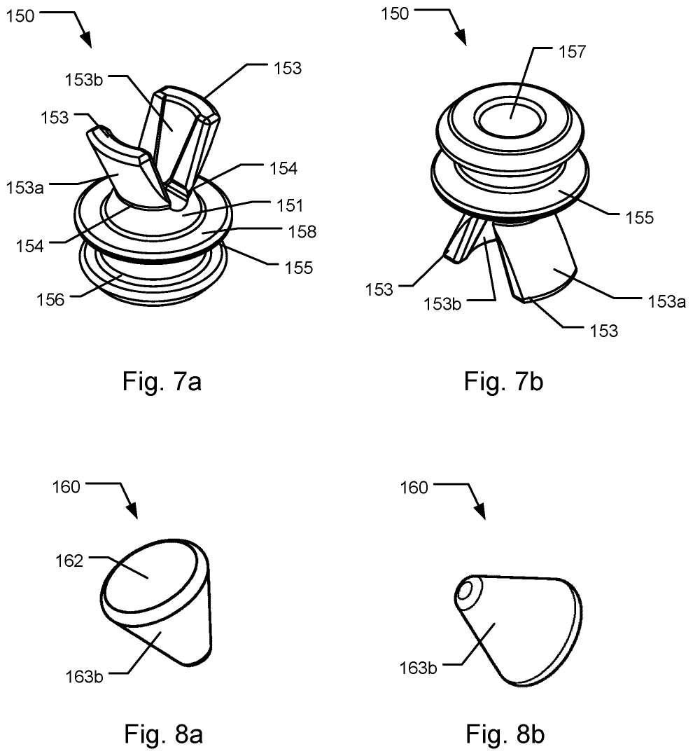

Comparing FIGS. 5a and 6 with FIGS. 1a and 2, which all show the capsules in the pre-actuation configuration, the overall design of the actuation is the same for the second embodiment device 100′ as compared with the first embodiment device 100, i.e. with a dissolvable latch support being formed as a cone and with latch arms having the same structure and function as described in the above. As shown in FIGS. 7a and 7b the hub 150 according to the second embodiment has been only slightly modified. Also, the upper sealing element 170 is similar. In addition, as shown in FIGS. 8a and 8b, the dissolvable latch support 160 is formed as a generally cone-shaped element.

In the second embodiment capsule device 100′, a lower sealing element 180 has been clamped between the upper capsule housing 110 and the lower capsule housing 120. In accordance herewith, the peripheral portion 181 of lower sealing element 180 is arranged slightly below a point axially midways between distal and proximal end portions of the capsule housing 110/120. In the shown embodiment, the first end 147 of the drive spring is mounted proximally to the lower sealing element 180 where it is clamped between spring seat portions arranged at the interface between the upper capsule housing 110 and the lower capsule housing 120. In the shown embodiment, the upper capsule housing 110 includes at its most distal portion an annular flange with a distally facing surface adapted to provide a seat for the first end of the drive spring 140.

The lower sealing element 180 additionally performs the function of guiding the payload portion 130, and more specifically guiding the tip portion of payload portion 130, as it travels during the delivery stroke, i.e. internally in the compartment of capsule device 100′ towards the exit hole 124. The seal element 180 again comprises a generally disc shaped structure and may be made of an elastic rubber-like material, such as silicone rubber. In the shown embodiment, in the pre-actuation configuration, a central portion 182 of sealing element 180 defines a conical portion sized and shaped to fit with the distal shape of payload portion 130 so as to provide a self-centring guide portion for the pointed tip of payload portion 130. As the sealing element 180 is made of elastic material, the material portions connecting the peripheral portion 181 with the central portion 182 forms a flexible connector 183 and allows the central portion 182 to be moved axially distally relative to the peripheral portion 181.

Initially, in the pre-actuation configuration, the sealing element 180 may provide a seal tight barrier preventing fluid externally to the compartment from entering into the compartment, thus protecting payload portion 130 from degradation. Resembling the first embodiment, the central portion 182 of the lower sealing element 180 may include one or more thin cuts, scores or similar weakened portions allowing the tip of payload portion 130 to force its way through the lower sealing element during the delivery stroke.

In FIG. 5b an intermediate state of the capsule device 100′ is depicted wherein the assembly consisting of the hub and the payload portion has moved distally so that the tip portion of payload portion 130 has moved to the axial position of the exit hole 124. In this embodiment, the tip portion has already at this point penetrated the lower sealing element 180. However, during its initial movement, the tip portion has been guided by the central portion 182 as the flexible connector 183 connecting the central portion 182 with the peripheral portion 181 has been stretched. In this state, the tension spring, i.e. drive spring 140, assumes a near flat configuration with the narrow second end 146 positioned only slightly proximal to the wide first end 147.

With the capsule device 100′ assuming the actuated configuration, refer to FIG. 5c, the hub 150 has moved all the way distally and halted by cooperation with proximally facing hub stop surface 128 arranged at the bottom part of capsule housing 120, and the payload portion has been moved into a position where the payload portion has penetrated and been inserted into tissue. The drive spring 140 assumes a configuration almost inverted relative to the initial pre-actuation configuration so that the narrow second end 146 is positioned distally to the wide first end 147 and in near proximity to the exit hole 124.

It is to be noted that further not shown embodiments in accordance with the invention may include actuator mechanisms having actuator interfaces formed differently than the conical shaped interface surfaces shown in connection with the first, second and third embodiments. For example, the actuator interfaces may be formed with planar surfaces instead of conical surfaces, either at the interface between the hub retainer structure 113 and the latch arm 153 and/or between the latch arm 153 and the dissolvable latch support 160. For such embodiment having two radially opposed latch arms, the dissolvable latch support 160 may be formed as a wedge having two planar surfaces intersecting each other at the sharp edge of the wedge.

Furthermore, the number of latch arms may be different than two, such as three, four or even more individual latch arms. In certain embodiments, the plurality of latch arms may be disposed equally around the actuation axis, although this may not be strictly necessary for any embodiment in accordance with the principles of the present invention.

The above described variants of interfaces between the payload portion 130 and the hub 150 are only exemplary and other configurations may be used instead. The detachable attachment between the payload portion and the hub may be obtained by using a friction or press fit. Alternatively, an adhesive may be used at the interface, such as sucrose. Still alternatively, the attachment may be obtained by initially wetting the payload portion and utilizing inherent stiction between the hub and the payload portion. In situation of use, upon the hub reaching its final destination, detachment may occur at the interface between the payload portion and the hub. In other embodiments, a desired detachment may be obtained by detaching a major portion of the payload portion from the remaining payload portion being still adhered or fastened to the hub. In some embodiments, the payload portion includes a weakened point which determines the point of separation. In still further embodiments, the hub and the payload portion may be formed as a unitary component all made of a composition containing API, and wherein the intended payload portion to be pushed out from capsule device is separated from the hub portion. Also, in alternative embodiments, the payload may act as a hub by itself to be fully transported away from the capsule device.

Although the above description of exemplary embodiments mainly concern ingestible capsules for delivery in the stomach, the present actuation principle generally finds utility in capsule devices for lumen insertion in general, wherein a capsule device is positioned into a body lumen, and wherein a fluid activates an actuation mechanism by dissolving a dissolvable latch support for bringing a component from a first configuration into a second configuration, such as from a first position into a second position. Non-limiting examples of capsule devices may include capsule devices for intestinal delivery of a payload or drug either by delivery into the intestinal lumen or into the tissue wall of an intestinal lumen. Drug delivery may be performed using a delivery member, such as a needle for injection of a liquid drug or powder, or via microneedles which is inserted into the tissue wall of a lumen, or which actuates directly into the lumen. Alternatively, drug delivery may be performed through one or more exit openings of the capsule device without the use of a delivery member, such as by jet injection of either a liquid drug or a particle stream into a mucosal lining of a lumen wall.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for ingestion for travelling into a lumen of the gastrointestinal tract of a patient, the lumen having a lumen wall, wherein the capsule device is configured as a self-righting capsule comprising:
a capsule housing comprising a compartment and an exterior tissue engaging surface defining an exit opening leading from the compartment to the capsule exterior,
a tissue penetrating member arranged along an axis and configured to be advanced axially from a first position in the compartment through the exit opening and into the lumen wall at a target location, and
an actuator for coupling to the tissue penetrating member, the actuator having a first configuration and a second configuration, wherein the actuator is configured, upon actuation, to move from the first configuration to the second configuration thereby advancing the tissue penetrating member axially from the first position and into the lumen wall,
wherein the actuator comprises a spring formed as a tapered helical tension spring, the spring having a wide first end coupled to the capsule housing and a narrow second end that couples to the tissue penetrating member, wherein, in the first configuration, accumulated tension in the spring is maintained, and wherein, upon actuation, accumulated tension in the spring is released thereby moving the actuator towards the second configuration.

2. The capsule device as in claim 1, wherein the tissue penetrating member has a distal end shaped for penetrating tissue and a proximal end coupled or coupleable to the actuator, wherein when the tissue penetrating member assumes the first position, the distal end is axially separated from the exit opening by a separating distance, thereby enabling the tissue penetrating member to be advanced towards the exit opening by an acceleration stroke corresponding to the separating distance.

3. The capsule device as in claim 2, wherein the acceleration stroke is between 1 mm and 8 mm, such as between 2 and 7 mm, and such as between 3 mm and 5 mm.

4. The capsule device as in claim 1, wherein a guide structure is arranged between the capsule housing and the tissue penetrating member, the guide structure comprising a central portion being configured for cooperating with the distal end of the tissue penetrating member thereby guiding the distal end for movement along the axis as the distal end of the tissue penetrating member moves towards the exit opening.

5. The capsule device as in claim 4, wherein the guide structure is configured for guiding the distal end of the tissue penetrating member along a major part of displacement of the acceleration stroke, such as more than 60 percent, such as more than 70 percent, such as more than 80 percent, and such as more than 90 percent of the acceleration stroke.

6. The capsule device as in claim 4, wherein the central portion is formed to engage with the tissue penetrating member and to allow the tissue penetrating member to escape through the central portion as the tissue penetrating member advances through the exit opening of the capsule housing.

7. The capsule device as in claim 4, wherein the guide structure comprises a peripheral portion coupled to the capsule housing, wherein the central portion is configured for axial movement, and wherein the guide structure comprises at least one flexible connector connecting the peripheral portion with the central portion.

8. The capsule device as in claim 7, wherein in the first configuration the at least one flexible connector assumes a shape having a portion that is inclined relative to the axis so as to extend from the peripheral portion to the central portion in a radially inwards and proximal direction.

9. The capsule device as in claim 8, wherein when the distal end of the tissue penetrating member assumes an axial position at the exit opening, said portion of the flexible connector has been shifted to assume a shape being inclined relative to the axis so as to extend from the peripheral portion to the central portion in a radially inwards and distal direction.

10. The capsule device as in claim 7, wherein the guide structure is formed at least in part from an elastomeric material, such as rubber, and wherein, in the first configuration, the at least one flexible connector is conically shaped.

11. The capsule device as in claim 1, wherein the spring in the first configuration assumes a conical shape extending along the axis.

12. The capsule device as in claim 1, wherein the spring is configured for moving an actuation member of the actuator in the distal direction and subsequentially in the proximal direction.

13. The capsule device as in claim 12, wherein the spring comprises a plurality of windings, and wherein, in the first configuration, at least one of the plurality of windings encircles the tissue penetrating member in an axially overlapping manner.

14. The capsule device as in claim 13, wherein the spring comprises a plurality of windings, and wherein, in the first configuration, a majority of the plurality of windings encircle the tissue penetrating member in an axially overlapping manner.

15. The capsule device as in claim 1, wherein the tissue penetrating member is a solid delivery member formed partly or entirely from a preparation comprising a therapeutic payload, and wherein the preparation is made from a dissolvable material that dissolves when inserted into tissue of the lumen wall to at least partially release the therapeutic payload into the blood stream.
